(19) United States
(12) Patent Application Publication
(10) Pub. No.: US 2025/0273323 A1
(43) Pub. Date: Aug. 28, 2025

(54) IMAGE GENERATION APPARATUS, IMAGE GENERATION METHOD, AND RECORDING MEDIUM

(71) Applicant: NEC Corporation, Tokyo (JP)

(72) Inventors: Hirokazu SAKAMOTO, Tokyo (JP); Tomoharu KIYUNA, Tokyo (JP); Tatsu KIMURA, Tokyo (JP)

(73) Assignee: NEC Corporation, Tokyo (JP)

(21) Appl. No.: 19/056,901

(22) Filed: Feb. 19, 2025

(30) Foreign Application Priority Data
Feb. 27, 2024 (JP) 2024-027606

(51) Int. Cl. G16H 30/40; G06T 7/00

(52) U.S. Cl. CPC G16H 30/40; G06T 7/0012; G06T 2207/30096

(57) ABSTRACT

This image generation apparatus includes: an acquiring section for acquiring a medical image of a patient; a detecting section for detecting a lesion candidate in the medical image; a differentiating section for generating, from information indicating the lesion candidate, a result of differentiation of the lesion candidate regarding a medical condition the patient has; an image generating section for generating, based on the information indicating the lesion candidate and the result of differentiation of the lesion candidate, a prediction image which represents a state in which the lesion candidate will be after a lapse of time; and an outputting section for outputting the prediction image.

### CROSS REFERENCE TO RELATED APPLICATIONS

This application is based upon and claims the benefit of priority from Japanese Patent Application No. 2024-027606 filed on Feb. 27, 2024, the disclosure of which is incorporated herein in its entirety by reference.

### TECHNICAL FIELD

The present disclosure relates to an image generation apparatus, an image generation method, and a recording medium.

### BACKGROUND ART

Diagnosis assist techniques are known. Examples of diagnosis assist techniques include the technique disclosed in Patent Literature 1. Patent Literature 1 discloses inputting a time series of a plurality of endoscopic images to a trained model which has learned endoscopic images and generating an endoscopic image at a next point (the next point of an endoscopic image that is located at the end of the time series).

### CITATION LIST

### Patent Literature

### [Patent Literature 1]

International Publication No. WO 2021/014584

### SUMMARY OF INVENTION

### Technical Problem

The technique disclosed in Patent Literature 1 is susceptible of improvement in terms of more accurately predicting changes in a lesion over time.

The present disclosure has been made in view of the above problem, and an example object thereof is to provide a technique which makes it possible to more accurately predict changes in a lesion over time.

### Solution to Problem

An image generation apparatus in accordance with an example aspect of the present disclosure includes at least one processor, and the at least one processor carries out: an acquiring process of acquiring a medical image of a patient; a detecting process of detecting a lesion candidate in the medical image; a differentiating process of generating, from information indicating the lesion candidate, a result of differentiation of the lesion candidate regarding a medical condition the patient has; an image generating process of generating, based on the information indicating the lesion candidate and the result of differentiation of the lesion candidate, a prediction image which represents a state in which the lesion candidate will be after a lapse of time; and an outputting process of outputting the prediction image.

An image generation method in accordance with an example aspect of the present disclosure includes: at least one processor acquiring a medical image of a patient; the at least one processor detecting a lesion candidate in the medical image; the at least one processor generating, from information indicating the lesion candidate, a result of differentiation of the lesion candidate regarding a medical condition the patient has; the at least one processor generating, based on the information indicating the lesion candidate and the result of differentiation of the lesion candidate, a prediction image which represents a state in which the lesion candidate will be after a lapse of time; and the at least one processor outputting the prediction image.

A recording medium in accordance with an example aspect of the present disclosure is a recording medium having recorded thereon a program for causing a computer to function as an image generation apparatus, and the program causes the computer to carry out: an acquiring process of acquiring a medical image of a patient; a detecting process of detecting a lesion candidate in the medical image; a differentiating process of generating, from information indicating the lesion candidate, a result of differentiation of the lesion candidate regarding a medical condition the patient has; an image generating process of generating, based on the information indicating the lesion candidate and the result of differentiation of the lesion candidate, a prediction image which represents a state in which the lesion candidate will be after a lapse of time; and an outputting process of outputting the prediction image.

### Advantageous Effects of Invention

An example aspect of the present disclosure provides an example advantage of making it possible to provide a technique for more accurately predict changes in a lesion over time.

### BRIEF DESCRIPTION OF DRAWINGS

FIG. 1 is a block diagram illustrating a configuration of an image generation apparatus in accordance with the present disclosure.

FIG. 2 is a flowchart illustrating a flow of an image generation method in accordance with the present disclosure.

FIG. 3 is a block diagram illustrating a configuration of an information processing apparatus in accordance with the present disclosure.

FIG. 4 is a diagram illustrating a functional configuration of a control section in accordance with the present disclosure.

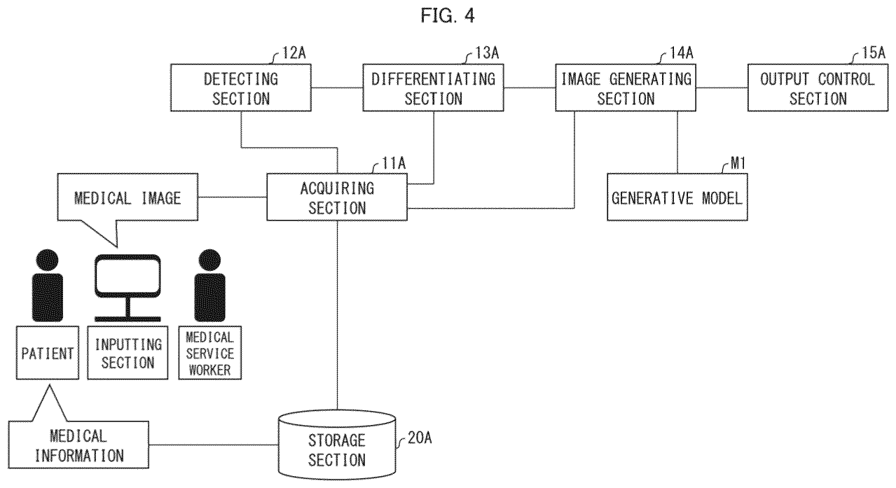

FIG. 5 is a representation of specific examples of a prediction image outputted by an output control section in accordance with the present disclosure.

FIG. 6 is a representation of specific examples of a prediction image outputted by the output control section in accordance with the present disclosure.

FIG. 7 is a flowchart illustrating an example flow of an image generation method in accordance with the present disclosure.

FIG. 8 is a block diagram illustrating a configuration of a computer which functions as the image generation apparatus or the information processing apparatus in accordance with the present disclosure.

### EXAMPLE EMBODIMENTS

The following description will discuss example embodiments of the present invention. However, the present invention is not limited to the example embodiments described below, but can be altered by a skilled person in the art within the scope of the claims. For example, any embodiment derived by appropriately combining techniques (some or all of products or methods) adopted in differing example embodiments described below can be within the scope of the present invention. Further, any embodiment derived by appropriately omitting one or more of the techniques adopted in differing example embodiments described below can be within the scope of the present invention. Furthermore, the advantage mentioned in each of the example embodiments described below is an example advantage expected in that example embodiment, and does not define the extension of the present invention. That is, any embodiment which does not provide any of the example advantages mentioned in the example embodiments described below can also be within the scope of the present invention.

### First Example Embodiment

The following description will discuss a first example embodiment, which is an example embodiment of the present invention, in detail with reference to the drawings. The present example embodiment is basic to each of the example embodiments which will be described later. It should be noted that the applicability of the techniques adopted in the present example embodiment is not limited to the present example embodiment. That is, the techniques adopted in the present example embodiment can be adopted in another example embodiment included in the present disclosure, to the extent of constituting no specific technical obstacle. Further, the techniques illustrated in the drawings referred to for the description of the present example embodiment can be adopted in another example embodiment included in the present disclosure, to the extent of constituting no specific technical obstacle.

### (Configuration of Image Generation Apparatus)

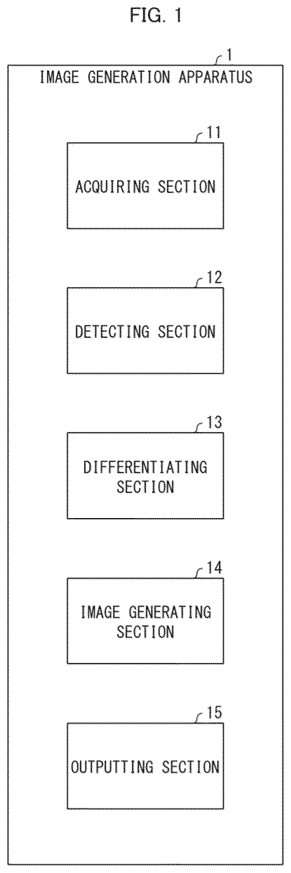

The configuration of an image generation apparatus 1 will be described below with reference to FIG. 1. FIG. 1 is a block diagram illustrating the configuration of the image generation apparatus 1. The image generation apparatus 1 includes an acquiring section 11, a detecting section 12, a differentiating section 13, an image generating section 14, and an outputting section 15, as illustrated in FIG. 1.

The acquiring section 11 acquires a medical image of a patient. The detecting section 12 detects a lesion candidate in the medical image. The differentiating section 13 generates, from information indicating the lesion candidate, a result of differentiation of the lesion candidate regarding a medical condition the patient has. The image generating section 14 generates, based on the information indicating the lesion candidate and the result of differentiation of the lesion candidate, a prediction image which represents a state in which the lesion candidate will be after a lapse of time. The outputting section 15 outputs the prediction image.

### (Example Advantage of Image Generation Apparatus)

As above, the image generation apparatus 1 includes: an acquiring section 11 for acquiring a medical image of a patient; a detecting section 12 for detecting a lesion candidate in the medical image; a differentiating section 13 for generating, from information indicating the lesion candidate, a result of differentiation of the lesion candidate regarding a medical condition the patient has; an image generating section 14 for generating, based on the information indicating the lesion candidate and the result of differentiation of the lesion candidate, a prediction image which represents a state in which the lesion candidate will be after a lapse of time; and an outputting section 15 for outputting the prediction image. Thus, the image generation apparatus 1 provides an example advantage of making it possible to more accurately predict changes in a lesion over time.

### (Flow of Image Generation Method)

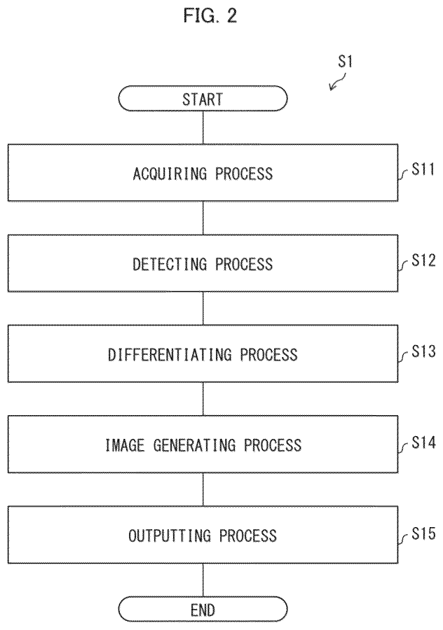

The flow of an image generation method S1 is described here with reference to FIG. 2. FIG. 2 is a flowchart illustrating the flow of the image generation method S1. The image generation method S1 includes an acquiring process S11, a detecting process S12, a differentiating process S13, an image generating process S14, and an outputting process S15, as illustrated in FIG. 2.

In the acquiring process S11, at least one processor acquires a medical image of a patient. In the detecting process S12, the at least one processor detects a lesion candidate in the medical image. In the differentiating process S13, the at least one processor generates, from information indicating the lesion candidate, a result of differentiation of the lesion candidate regarding a medical condition the patient has. In the image generating process S14, the at least one processor generates, based on the information indicating the lesion candidate and the result of differentiation of the lesion candidate, a prediction image which represents a state in which the lesion candidate will be after a lapse of time. In the outputting process S15, the at least one processor outputs the prediction image.

### (Example Advantage of Image Generation Method)

As above, the image generation method S1 includes: an acquiring process S11 of at least one processor acquiring a medical image of a patient; a detecting process S12 of the at least one processor detecting a lesion candidate in the medical condition; a differentiating process S13 of the at least one processor generating, from information indicating the lesion candidate, a result of differentiation of the lesion candidate regarding a medical condition the patient has; an image generating process S14 of the at least one processor generating, based on the information indicating the lesion candidate and the result of differentiation of the lesion candidate, a prediction image which represents a state in which the lesion candidate will be after a lapse of time; and an outputting process S15 of the at least one processor outputting the prediction image. Thus, the image generation method S1 provides an example advantage of making it possible to more accurately predict changes in a lesion over time.

### Second Example Embodiment

The following description will discuss a second example embodiment, which is an example embodiment of the present invention, in detail with reference to the drawings. A component having the same function as a component described in the above example embodiment is assigned the same reference sign, and the description thereof is omitted where appropriate. It should be noted that the applicability of the techniques adopted in the present example embodiment is not limited to the present example embodiment. That is, the techniques adopted in the present example embodiment can be adopted in another example embodiment included in the present disclosure, to the extent of constituting no specific technical obstacle. Further, the techniques illustrated in the drawings referred to for the description of the present example embodiment can be adopted in another example embodiment included in the present disclosure, to the extent of constituting no specific technical obstacle.

### (Configuration of Information Processing Apparatus)

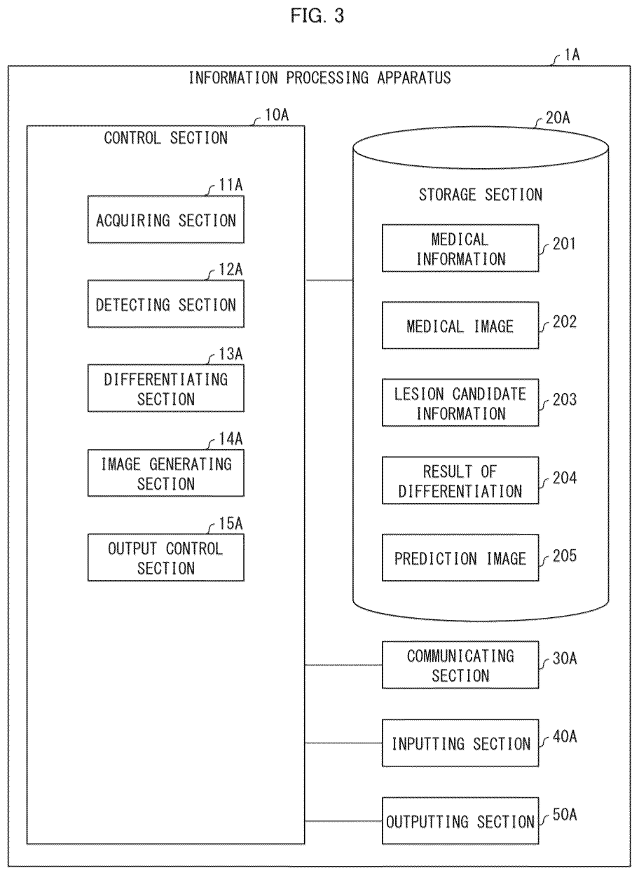

The configuration of an information processing apparatus 1A is described here with reference to FIG. 3. FIG. 3 is a block diagram illustrating the configuration of the information processing apparatus 1A. The information processing apparatus 1A includes a control section 10A, a storage section 20A, a communicating section 30A, an inputting section 40A, and an outputting section 50A.

### (Communicating Section)

The communicating section 30A communicates with an apparatus external to the information processing apparatus 1A over a communication line. A specific configuration of the communication line does not limit the present example embodiment, but examples of the communication line include a wireless local area network (LAN), a wired LAN, a wide area network (WAN), a public network, a mobile data communication network, and a combination thereof. The communicating section 30A transmits, to another apparatus, data supplied from the control section 10A, and supplies the control section 10A with data received from another apparatus.

### (Inputting Section)

The inputting section 40A is a component for accepting an input to the information processing apparatus 1A, and includes inputting equipment such as, for example, an endoscope, a keyboard, a mouse, a touch panel, a camera, or a microphone. The inputting section 40A is configured to receive endoscopic video or signal from an endoscope. Further, the inputting section 40A may be configured to accept data from the inputting equipment via an interface such as, for example, a universal serial bus (USB).

### (Outputting Section)

The outputting section 50A is a component for producing an output from the information processing apparatus 1A, and includes outputting equipment such as, for example, a display, a printer, a touch panel, or a speaker. The outputting section 50A may be configured to include an interface such as, for example, a USB and output data to the outputting equipment via the interface.

### (Storage Section)

In the storage section 20A, various kinds of information to be referred to by the control section 10A are stored. Examples of such information include medical information 201, a medical image 202, lesion candidate information 203, a result 204 of differentiation, and a prediction image 205.

### (Medical Information)

The medical information 201 is information regarding medical care for a patient. Examples of the medical information 201 include medical record information. As an example, the medical information 201 includes at least one selected from the group consisting of personal information regarding the patient, information on findings shown by a medical examination performed on the patient, and medical history information regarding the patient. The personal information regarding the patient is personal information regarding the patient, and includes, for example, information indicating the age, the gender, the amount of smoking, etc. of the patient. The information on findings shown by a medical examination performed on the patient is information indicating findings made by a medical service worker, such as a doctor, and includes, for example, information obtained by the medical service worker through a medical interview or an inspection. The information obtained through a medical interview or an inspection may be, for example, text “pressure on the abdomen causes a pain”. The medical history information regarding the patient is information regarding the history of disease of the patient, and includes, for example, information indicating the past medical history of the patient, the past medical history of the family, the amount of smoking of the patient, etc. In addition, the medical information 201 may include information indicating the measurement results of vital signs of the patient. The information indicating the measurement results of vital signs may be, for example, information indicating “37.8° C./heart rate 90/breathing rate 30”.

### (Medical Image)

The medical image 202 is an image of a patient to be used for diagnosis and therapy. The medical image 202 includes an endoscopic image. Further, the medical image 202 may include at least one of images which are an X-ray image, an endoscopic image, a pathological image, an MRI image, and a CT image. The storage section 20A stores the respective pieces of medical information 201 of a plurality of patients and the respective medical images 202 of the plurality of patients.

### (Lesion Candidate Information)

The lesion candidate information 203 is information indicating a lesion candidate which is detected in the medical image 202. For example, the lesion candidate information 203 is information (such as coordinate information) which indicates a lesion region or image data which represents an image obtained by marking the location of the lesion candidate in the medical image 202. Further, the lesion candidate information 203 may be data in text format.

### (Result of Differentiation)

The result 204 of differentiation is a result of differentiation of the lesion candidate regarding a medical condition a patient has. As used herein, the result of differentiation of the lesion candidate refers to a pseudo diagnosis candidate generated by a differentiating section 13A. The result of differentiation of the lesion candidate is, for example, the result of qualitative diagnosis (classification into neoplastic/non-neoplastic types). Further, the result of differentiation of the lesion candidate may include information which indicates the name of a disease. Examples of the result 204 of differentiation of the lesion candidate includes, but are not limited to, data in text format.

### (Prediction Image)

The prediction image 205 is an image which represents a state in which the lesion candidate indicated by the lesion candidate information 203 will be after changing over time. It can be said that the prediction image 205 is an image which represents a prediction of a change in a lesion over time. As an example, the prediction image 205 is used in decision making carried out by a medical service worker who provides medical examination and treatment.

### (Control Section)

The control section 10A includes an acquiring section 11A, a detecting section 12A, a differentiating section 13A, an image generating section 14A, and an output control section 15A.

### (Acquiring Section)

The acquiring section 11A acquires the medical image 202 of a patient to be diagnosed. The acquiring section 11A acquires, via the inputting section 40A, endoscopic video formed by a plurality of still images or a moving image, and acquires, as the medical image 202, a plurality of still images contained in the endoscopic video. Further, as an example, the acquiring section 11A may acquire the medical image 202 by retrieving the medical image 202 from a storage location (which may be storage in the information processing apparatus 1A, or may be storage external to the information processing apparatus 1A) designated by a user of the information processing apparatus 1A. The acquiring section 11A may acquire the medical image 202 by receiving the medical image 202 from another apparatus via the communicating section 30A. The acquiring section 11A may acquire the medical image 202 inputted to the inputting section 40A.

The acquiring section 11A may acquire the medical information 201 in addition to the medical image 202. In this case, the acquiring section 11A may acquire the medical information 201 by retrieving the medical information 201 from a storage location (which may be storage in the information processing apparatus 1A, or may be storage external to the information processing apparatus 1A) designated by a user of the information processing apparatus 1A. The acquiring section 11A may acquire the medical information 201 by receiving the medical information 201 from another apparatus via the communicating section 30A. The acquiring section 11A may acquire the medical information 201 inputted to the inputting section 40A.

### (Detecting Section)

The detecting section 12A detects a lesion candidate in the medical image 202. As an example, the detecting section 12A uses a detection model for detecting a lesion in an image, to detect a lesion candidate in the medical image 202. The detection model may be stored in the storage section 20A of the information processing apparatus 1A, or may be stored in an apparatus other than the information processing apparatus 1A. The detection model being stored in the storage section 20A means that parameters defining the detection model are stored in the storage section 20A.

As an example, the detection model is a trained model generated by machine learning. Examples of the detection model include, but are not limited to, a trained neural network or the like which is obtained by learning in which a plurality of training images are referred to.

As an example, an input to the detection model is the medical image 202. Further, an input to the detection model may include information other than the medical image 202. As an example, an output from the detection model is the lesion candidate information 203, which indicates a lesion candidate. Further, an output from the detection model may include information other than the lesion candidate information 203.

In a case where the detection model is stored in an apparatus other than the information processing apparatus 1A, the detecting section 12A inputs the medical image 202 to the detection model by, for example, transmitting the medical image 202 via the communicating section 30A to the apparatus which has stored therein the detection model. In this case, the detecting section 12A receives the information outputted by the detection model from that apparatus via the communicating section 30A.

### (Differentiating Section)

The differentiating section 13A generates a result of differentiation of a lesion candidate regarding a medical condition a patient has. As an example, the differentiating section 13A uses a differentiation model generated by machine learning, to generate a result of differentiation of a lesion candidate. The differentiation model may be stored in the storage section 20A of the information processing apparatus 1A, or may be stored in an apparatus other than the information processing apparatus 1A. The differentiation model being stored in the storage section 20A means that parameters defining the differentiation model are stored in the storage section 20A.

As an example, the differentiation model is a trained model generated by machine learning. Examples of the differentiation model include, but are not limited to: a trained model generated by supervised learning with use of a neural network approach or the like; and a model obtained by fine-tuning, with medical information, generative AI such as Chat Generative Pre-trained Transformer (ChatGPT) or Generative Pre-trained Transformer 4 (GPT-4).

As an example, input information to be inputted by the differentiating section 13A to the differentiation model includes the lesion candidate information 203. Further, the input information may include the medical information 201 regarding a patient. In a case where the lesion candidate information 203 is coordinate information, the differentiating section 13A may use the medical image 202 and the coordinate information to generate an image (e.g., an image in which the location of a lesion candidate is marked) which represents the lesion candidate, and include the generated image in the input information.

An output from the differentiation model is information which indicates a result of differentiation of a lesion candidate. The result of differentiation of a lesion candidate may be, for example, information which indicates “neoplastic” or “non-neoplastic”.

In a case where the differentiation model is stored in an apparatus other than the information processing apparatus 1A, the differentiating section 13A inputs the input information to the detection model by, for example, transmitting the input information via the communicating section 30A to the apparatus which has stored therein the differentiation model. In this case, the differentiating section 13A acquires information outputted by the differentiation model, by receiving the information from that apparatus via the communicating section 30A.

The differentiating section 13A may generate results 204 of differentiation of a plurality of lesion candidates from the lesion candidate information 203. The differentiating section 13A may generate the results 204 of differentiation of a plurality of lesion candidates by, for example, using a plurality of differentiation models different from each other.

### (Image Generating Section)

The image generating section 14A generate, based on the lesion candidate information 203 and the result 204 of differentiation of the lesion candidate, the prediction image 205, which represents a state in which the lesion candidate will be after changing over time. As an example, the image generating section 14A uses a generative model M1 generated by machine learning, to generate the prediction image 205 based on the lesion candidate information 203 and the result 204 of differentiation of the lesion candidate.

As an example, the generative model M1 is a model which is trained using a result of a lapse of time on a lesion as ground truth data, so as to generate a prediction image that represents a state in which a lesion will be after changing over time. Examples of the generative model M1 include, but are not limited to, a model obtained by fine-tuning, with medical information, generative AI such as ChatGPT or GPT-4.

Input information to be inputted by the image generating section 14A to the generative model M1 includes the lesion candidate information 203 and the result 204 of differentiation of the lesion candidate. Further, the input information may include the medical information 201. In this case, in other words, it can be said that the acquiring section 11A acquires medical information, and the image generating section 14A inputs the lesion candidate information 203, the result 204 of differentiation of the lesion candidate, and the medical information 201 to the generative model M1 to generate the prediction image 205.

As an example, an output from the generative model M1 includes the prediction image 205. Further, an output from the generative model M1 may include the accuracy of the prediction image 205. In this case, in other words, it can be said that the image generating section 14A uses the generative model M1 to generate the accuracy of the prediction image 205.

In a case where the generative model M1 is stored in an apparatus other than the information processing apparatus 1A, the image generating section 14A inputs the input information to the generative model M1 by, for example, transmitting the input information via the communicating section 30A to the apparatus which has stored therein the generative model M1. In this case, the image generating section 14A receives the prediction image 205 outputted by the generative model M1 from that apparatus via the communicating section 30A.

### (Generation of Plurality of Prediction Images)

The image generating section 14A may generate a plurality of prediction images 205. As an example, the image generating section 14A may use a plurality of generative models different from each other, to generate the plurality of prediction images 205. Further, the image generating section 14A may generate the prediction images 205 by inputting, to the generative model M1, a plurality of pieces of input information which are different from each other. In this case, for example, the plurality of pieces of input information may differ from each other partially or wholly in terms of the medical information 201 contained therein. In a case where the differentiating section 13A generates the results 204 of differentiation of the plurality of lesion candidates, the image generating section 14A may generate respective prediction images 205 for the results 204 of differentiation of the plurality of lesion candidates, which are generated by the differentiating section 13A. In a case where the image generating section 14A generates the plurality of prediction images 205, the image generating section 14A may generate the respective accuracies of the plurality of prediction images 205.

### (Output Control Section)

The output control section 15A outputs one or more prediction images 205. As an example, the output control section 15A may output the prediction images 205 by writing the prediction images 205 in a storage location (which may be storage in the information processing apparatus 1A, or may be storage external to the information processing apparatus 1A) designated by a user of the information processing apparatus 1A. The output control section 15A may output the prediction images 205 by transmitting the prediction images 205 via the communicating section 30A, or may output the prediction images 205 to outputting equipment such as a display. Further, the output control section 15A may output the plurality of prediction images 205 and the respective accuracies of the prediction images 205.

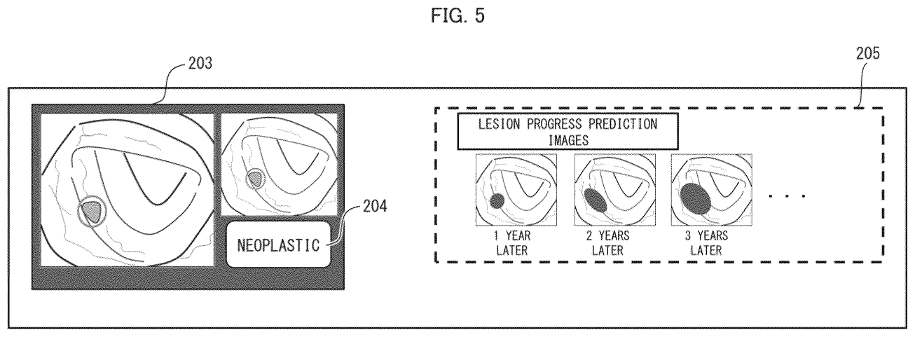

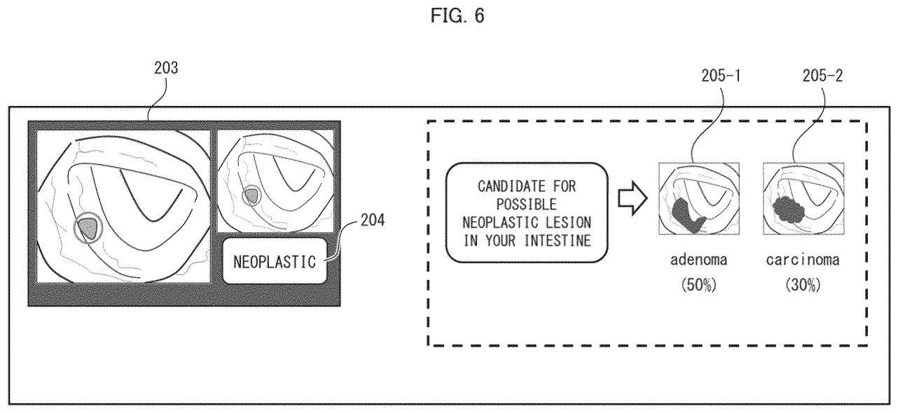

FIGS. 5 and 6 are representations of specific examples of the prediction image 205 outputted by the output control section 15A. As an example, the output control section 15A may display the lesion candidate information 203, the result 204 of differentiation of the lesion candidate, and the prediction image 205 on displaying equipment, as illustrated in FIG. 5. In the example of FIG. 5, output information outputted by the output control section 15A includes: the lesion candidate information 203 that is a medical image in which the location of a lesion is marked; and the result 204 of differentiation of the lesion candidate. The output information further includes respective prediction images of the lesion which is predicted to become 1 year later, 2 years later, 3 years later, and so on, as progress prediction images of the lesion.

In the examples of FIG. 6, the output information outputted from the output control section 15A includes prediction images 205-1 and 205-2 corresponding to respective results of differentiation of different lesion candidates. More specifically, displayed in the example of FIG. 6 are: the prediction image 205-1 and the accuracy thereof for the case where the “candidate for a possible neoplastic lesion in your intestine” is “adenoma”; and the prediction image 205-2 and the accuracy thereof for the case of “carcinoma”. A medical service worker or the like who provides a patient with medical examination and treatment can use the prediction image 205 outputted by the output control section 15A as, for example, the basis for decision making on the strategy for therapy.

### (Flow of Image Generation Method)

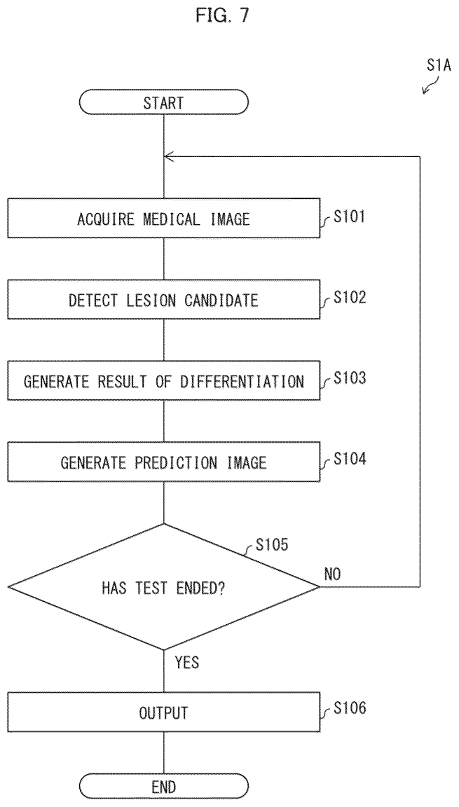

FIG. 7 is a flowchart illustrating an example flow of an image generation method S1A carried out by the information processing apparatus 1A. The steps included in the flowchart of FIG. 7 may be carried out in parallel with each other or in a different order. In step S101, the acquiring section 11A acquires the medical image 202. In step S102, the detecting section 12A detects a lesion candidate in the medical image 202. In step S103, the differentiating section 13A uses the lesion candidate information 203 to generate the result 204 of differentiation of the lesion candidate regarding a medical condition a patient has. In step S104, the image generating section 14A generates the prediction image 205, which represents a state in which the lesion candidate will be after a lapse of time.

In step S105, the image generating section 14A determines whether a test has ended. For example, this determination may be made by determining whether the instructions to the effect that the test should be ended have been inputted by a medical service worker or the like to the information processing apparatus 1A. In a case where the test is ended (YES in step S105), the image generating section 14A moves to the process of step S106. In step S106, the output control section 15A outputs the prediction image 205 generated in step S104. In a case of continuing with the test (NO in step S105), the image generating section 14A moves to the process of step S101. In this manner, the information processing apparatus 1A carries out the processes of steps S101 to S104 repeatedly until the test ends.

### (Example Advantage of Information Processing Apparatus)

As above, the information processing apparatus 1A includes: an acquiring section 11A for acquiring the medical image 202 of a patient; a detecting section 12A for detecting a lesion candidate in the medical image 202; a differentiating section 13A for generating, from the lesion candidate information 203, the result 204 of differentiation of the lesion candidate regarding a medical condition the patient has; an image generating section 14A for generating, based on the lesion candidate information 203 and the result 204 of differentiation of the lesion candidate, the prediction image 205 which represents a state in which the lesion candidate will be after a lapse of time; and an output control section 15A for outputting the prediction image 205. By generating the prediction image 205 with use of not only the lesion candidate information 203 but also the result 204 of differentiation of a lesion candidate, the information processing apparatus 1A provides an example advantage of making it possible to more accurately predict changes in a lesion over time.

In the information processing apparatus 1A, the image generating section 14A generates, based on the lesion candidate information 203 and the result 204 of differentiation of the lesion candidate, a plurality of prediction images 205 which each represent a state in which the lesion candidate will be after a lapse of time. Thus, the information processing apparatus 1A provides an example advantage of making it possible to predict a plurality of states, which are future states of the lesion, from the medical image 202 of a patient.

In the information processing apparatus 1A, the image generating section 14A generates the respective accuracies of the plurality of prediction images 205, and the output control section 15A outputs the plurality of prediction images 205 and the respective accuracies of the prediction images 205. Thus, the information processing apparatus 1A provides an example advantage of making it possible to not only understand a plurality of states, which are predicted future states of a lesion, but also understand the respective accuracies of the plurality of states, by a medical service worker or the like checking the outputted prediction images 205 and the outputted respective accuracies of the prediction images 205, the medical service worker or the like providing a patient with medical examination and treatment.

In the information processing apparatus 1A, the differentiating section 13A generates the results 204 of differentiation of a plurality of lesion candidates from the lesion candidate information 203, and the image generating section 14A generates a prediction image 205 for each of the results 204 of differentiation of the plurality of lesion candidates. Thus, the information processing apparatus 1A provides an example advantage of making it possible for a medical service worker or the like who provides a patient with medical examination and treatment to understand the respective prediction images 205 corresponding to the results 204 of differentiation of the plurality of lesion candidates.

In the information processing apparatus 1A, the image generating section 14A generates the prediction image 205, based on the lesion candidate information 203 and the result 204 of differentiation of the lesion candidate, with use of the generative model M1 trained by machine learning in which a result of a lapse of time on a lesion is used as ground truth data, so as to generate a prediction image that represents a state in which the lesion will be after a lapse of time. Thus, the information processing apparatus 1A provides an example advantage of making it possible to more accurately predict changes in a lesion over time.

In the information processing apparatus 1A, the acquiring section 11A acquires the medical information 201 regarding a patient in addition to the medical image 202, and the image generating section 14A inputs the lesion candidate information 203, the result 204 of differentiation of the lesion candidate, and the medical information 201 to the generative model M1, to generate the prediction image 205. Thus, the information processing apparatus 1A provides an example advantage of making it possible to more accurately predict changes in a lesion over time, by using the medical information 201 in addition to the lesion candidate information 203 and the result 204 of differentiation of the lesion candidate.

In the information processing apparatus 1A, the medical information 201 includes at least one selected from the group consisting of personal information regarding a patient, information on findings shown by a medical examination performed on the patient, and medical history information regarding the patient. Thus, the information processing apparatus 1A provides an example advantage of making it possible to more accurately predict changes in a lesion over time, by using at least one selected from the group consisting of personal information regarding a patient, information on findings shown by a medical examination performed on the patient, and medical history information regarding the patient.

In the information processing apparatus 1A, the prediction image 205 is used in decision making carried out by a medical service worker who provides medical examination and treatment. Thus, the information processing apparatus 1A makes it possible for a medical service worker who provides medical examination and treatment to more properly carry out decision making.

### [Software Implementation Example]

Some or all of the functions of the image generation apparatus 1 and the information processing apparatus 1A (hereinafter, also referred to as “each apparatus above”) may be implemented by hardware such as an integrated circuit (IC chip), or may be implemented by software.

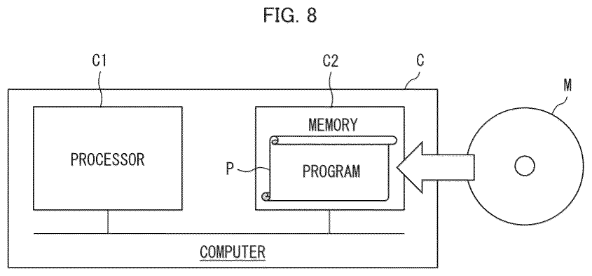

In the latter case, each apparatus above is provided by, for example, a computer that executes instructions of a program that is software implementing the functions. An example (hereinafter, computer C) of such a computer is illustrated in FIG. 8. FIG. 8 is a block diagram illustrating a hardware configuration of the computer C which functions as each apparatus above.

The computer C includes at least one processor C1 and at least one memory C2. The memory C2 has recorded thereon a program P for causing the computer C to operate as each apparatus above. The processor C1 of the computer C retrieves the program P from the memory C2 and executes the program P, so that the functions of each apparatus above are implemented.

Examples of the processor C1 can include a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, and a combination thereof. Examples of the memory C2 can include a flash memory, a hard disk drive (HDD), a solid state drive (SSD), and a combination thereof.

The computer C may further include a random access memory (RAM) into which the program P is loaded at the time of execution and in which various kinds of data are temporarily stored. The computer C may further include a communication interface via which data is transmitted to and received from another apparatus. The computer C may further include an input-output interface via which inputting-outputting equipment such as a keyboard, a mouse, a display, or a printer is connected.

The program P can be recorded on a non-transitory tangible recording medium M capable of being read by the computer C. The recording medium M can be, for example, a tape, a disk, a card, a semiconductor memory, a programmable logic circuit, or the like. The computer C can obtain the program P via such a recording medium M. The program P can be transmitted via a transmission medium. Examples of such a transmission medium can include a communication network and a broadcast wave. The computer C can obtain the program P also via such a transmission medium.

The above-described functions of each apparatus above may be implemented by a single processor provided in a single computer, may be implemented by the cooperation among a plurality of processors provided in a single computer, or may be implemented by the cooperation among a plurality of processors provided in a plurality of respective computers. Further, the program for causing each apparatus above to implement the above-described functions may be stored in a single memory provided in a single computer, may be stored in a distributed manner in a plurality of memories provided in a single computer, or may be stored in a distributed manner in a plurality of memories provided in a plurality of respective computers.

### Additional Remark 1

The whole or part of the example embodiments disclosed above can be described as, but not limited to, the following supplementary notes, and the present invention can be altered in various ways by a skilled person in the art within the scope of the claims

### Additional Remark a

The whole or part of the example embodiments disclosed above can be described as, but not limited to, the following supplementary notes, and the present invention can be altered in various ways by a skilled person in the art within the scope of the claims

### Supplementary Note A1

An image generation apparatus, including: an acquiring means for acquiring a medical image of a patient;
a detecting means for detecting a lesion candidate in the medical image;
a differentiating means for generating, from information indicating the lesion candidate, a result of differentiation of the lesion candidate regarding a medical condition the patient has;
an image generating means for generating, based on the information indicating the lesion candidate and the result of differentiation of the lesion candidate, a prediction image which represents a state in which the lesion candidate will be after a lapse of time; and
an outputting means for outputting the prediction image.

### Supplementary Note A2

The image generation apparatus described in supplementary note A1, in which the image generating means is configured to generate, based on the information indicating the lesion candidate and the result of differentiation of the lesion candidate, a plurality of prediction images which each represent a state in which the lesion candidate will be after a lapse of time.

### Supplementary Note A3

The image generation apparatus described in supplementary note A2, in which the image generating means is configured to generate respective accuracies of the plurality of prediction images; and
the outputting means is configured to output the plurality of prediction images and the respective accuracies of the plurality of prediction images.

### Supplementary Note A4

The image generation apparatus described in supplementary note A2 or A3, in which the differentiating means is configured to generate results of differentiation of a plurality of lesion candidates from the information indicating the lesion candidate; and
the image generating means is configured to generate respective prediction images for the results of differentiation of the plurality of lesion candidates.

### Supplementary Note A5

The image generation apparatus described in any one of supplementary notes A1 to A4, in which the image generating means is configured to generate the prediction image based on the information indicating the lesion candidate and the result of differentiation of the lesion candidate, with use of a generative model trained by machine learning in which a result of a lapse of time on a lesion is used as ground truth data, so as to generate a prediction image that represents a state in which the lesion will be after a lapse of time.

### Supplementary Note A6

The image generation apparatus described in supplementary note A5, in which the acquiring means is configured to acquire medical information regarding the patient in addition to the medical image; and
the image generating means is configured to generate the prediction image by inputting the information indicating the lesion candidate, the result of differentiation of the lesion candidate, and the medical information to the generative model.

### Supplementary Note A7

The image generation apparatus described in supplementary note A6, in which the medical information includes at least one selected from the group consisting of personal information regarding the patient, information on findings shown by a medical examination performed on the patient, and medical history information regarding the patient.

### Supplementary Note A8

The image generation apparatus described in any one of supplementary notes A1 to A7, in which the prediction image is used in decision making carried out by a medical service worker who provides medical examination and treatment.

### Additional Remark B

The whole or part of the example embodiments disclosed above can be described as, but not limited to, the following supplementary notes, and the present invention can be altered in various ways by a skilled person in the art within the scope of the claims

### Supplementary Note B1

An image generation method, including: at least one processor acquiring a medical image of a patient;
the at least one processor detecting a lesion candidate in the medical image;
the at least one processor generating, from information indicating the lesion candidate, a result of differentiation of the lesion candidate regarding a medical condition the patient has;
the at least one processor generating, based on the information indicating the lesion candidate and the result of differentiation of the lesion candidate, a prediction image which represents a state in which the lesion candidate will be after a lapse of time; and
the at least one processor outputting the prediction image.

### Supplementary Note B2

The image generation method described in supplementary note B1, in which in the generating of the prediction image, the at least one processor generates, based on the information indicating the lesion candidate and the result of differentiation of the lesion candidate, a plurality of prediction images which each represent a state in which the lesion candidate will be after a lapse of time.

### Supplementary Note B3

The image generation method described in supplementary note B2, in which in the generating of the prediction image, the at least one processor generates respective accuracies of the plurality of prediction images; and
in the outputting, the at least one processor outputs the plurality of prediction images and the respective accuracies of the plurality of prediction images.

### Supplementary Note B4

The image generation method described in supplementary note B2 or B3, in which in the generating of the result of differentiation, the at least one processor generates results of differentiation of a plurality of lesion candidates from the information indicating the lesion candidate; and
in the generating of the prediction image, the at least one processor generates respective prediction images for the results of differentiation of the plurality of lesion candidates.

### Supplementary Note B5

The image generation method described in any one of supplementary notes B1 to B4, in which in the generating of the prediction image, the at least one processor generates the prediction image based on the information indicating the lesion candidate and the result of differentiation of the lesion candidate, with use of a generative model trained by machine learning in which a result of a lapse of time on a lesion is used as ground truth data, so as to generate a prediction image that represents a state in which the lesion will be after a lapse of time.

### Supplementary Note B6

The image generation method described in supplementary note B5, in which in the acquiring, the at least one processor acquires medical information regarding the patient in addition to the medical image; and
in the generating of the prediction image, the at least one processor generates the prediction image by inputting the information indicating the lesion candidate, the result of differentiation of the lesion candidate, and the medical information to the generative model.

### Supplementary Note B7

The image generation method described in supplementary note B6, in which the medical information includes at least one selected from the group consisting of personal information regarding the patient, information on findings shown by a medical examination performed on the patient, and medical history information regarding the patient.

### Supplementary Note B8

The image generation method described in any one of supplementary notes B1 to B7, in which the prediction image is used in decision making carried out by a medical service worker who provides medical examination and treatment.

### Additional Remark C

The whole or part of the example embodiments disclosed above can be described as, but not limited to, the following supplementary notes, and the present invention can be altered in various ways by a skilled person in the art within the scope of the claims

### Supplementary Note C1

An image generation program for causing a computer to function as an image generation apparatus,
the program causing the computer to function as:
an acquiring means for acquiring a medical image of a patient;
a detecting means for detecting a lesion candidate in the medical image;
a differentiating means for generating, from information indicating the lesion candidate, a result of differentiation of the lesion candidate regarding a medical condition the patient has;
an image generating means for generating, based on the information indicating the lesion candidate and the result of differentiation of the lesion candidate, a prediction image which represents a state in which the lesion candidate will be after a lapse of time; and
an outputting means for outputting the prediction image.

### Supplementary Note C2

The image generation program described in supplementary note C1, in which the image generating means is configured to generate, based on the information indicating the lesion candidate and the result of differentiation of the lesion candidate, a plurality of prediction images which each represent a state in which the lesion candidate will be after a lapse of time.

### Supplementary Note C3

The image generation program described in supplementary note C2, in which the image generating means is configured to generate respective accuracies of the plurality of prediction images; and
the outputting means is configured to output the plurality of prediction images and the respective accuracies of the plurality of prediction images.

### Supplementary Note C4

The image generation program described in supplementary note C2 or C3, in which the differentiating means is configured to generate results of differentiation of a plurality of lesion candidates from the information indicating the lesion candidate; and
the image generating means is configured to generate respective prediction images for the results of differentiation of the plurality of lesion candidates.

### Supplementary Note C5

The image generation program described in any one of supplementary notes C1 to C4, in which the image generating means is configured to generate the prediction image based on the information indicating the lesion candidate and the result of differentiation of the lesion candidate, with use of a generative model trained by machine learning in which a result of a lapse of time on a lesion is used as ground truth data, so as to generate a prediction image that represents a state in which the lesion will be after a lapse of time.

### Supplementary Note C6

The image generation program described in supplementary note C5, in which the acquiring means is configured to acquire medical information regarding the patient in addition to the medical image; and
the image generating means is configured to generate the prediction image by inputting the information indicating the lesion candidate, the result of differentiation of the lesion candidate, and the medical information to the generative model.

### Supplementary Note C7

The image generation program described in supplementary note C6, in which the medical information includes at least one selected from the group consisting of personal information regarding the patient, information on findings shown by a medical examination performed on the patient, and medical history information regarding the patient.

### Supplementary Note C8

The image generation program described in any one of supplementary notes C1 to C7, in which the prediction image is used in decision making carried out by a medical service worker who provides medical examination and treatment.

### Additional Remark D

The whole or part of the example embodiments disclosed above can be described as, but not limited to, the following supplementary notes, and the present invention can be altered in various ways by a skilled person in the art within the scope of the claims

### Supplementary Note D1

An image generation apparatus, including at least one processor, the at least one processor carrying out:
an acquiring process of acquiring a medical image of a patient;
a detecting process of detecting a lesion candidate in the medical image;
a differentiating process of generating, from information indicating the lesion candidate, a result of differentiation of the lesion candidate regarding a medical condition the patient has;
an image generating process of generating, based on the information indicating the lesion candidate and the result of differentiation of the lesion candidate, a prediction image which represents a state in which the lesion candidate will be after a lapse of time; and an outputting process of outputting the prediction image.

The image generation apparatus may further include a memory. The memory may have stored therein a program for causing the at least one processor to carry out each of the processes.

### Supplementary Note D2

The image generation apparatus described in supplementary note D1, in which in the image generating process, the at least one processor generates, based on the information indicating the lesion candidate and the result of differentiation of the lesion candidate, a plurality of prediction images which each represent a state in which the lesion candidate will be after a lapse of time.

### Supplementary Note D3

The image generation apparatus described in supplementary note D2, in which in the image generating process, the at least one processor generates respective accuracies of the plurality of prediction images; and
in the outputting process, the at least one processor outputs the plurality of prediction images and the respective accuracies of the plurality of prediction images.

### Supplementary Note D4

The image generation apparatus described in supplementary note D2 or D3, in which in the differentiating process, the at least one processor generates results of differentiation of a plurality of lesion candidates from the information indicating the lesion candidate; and
in the image generating process, the at least one processor generates respective prediction images for the results of differentiation of the plurality of lesion candidates.

### Supplementary Note D5

The image generation apparatus described in any one of supplementary notes D1 to D4, in which in the image generating process, the at least one processor generates the prediction image based on the information indicating the lesion candidate and the result of differentiation of the lesion candidate, with use of a generative model trained by machine learning in which a result of a lapse of time on a lesion is used as ground truth data, so as to generate a prediction image that represents a state in which the lesion will be after a lapse of time.

### Supplementary Note D6

The image generation apparatus described in supplementary note D5, in which in the acquiring process, the at least one processor acquires medical information regarding the patient in addition to the medical image; and
in the image generating process, the at least one processor generates the prediction image by inputting the information indicating the lesion candidate, the result of differentiation of the lesion candidate, and the medical information to the generative model.

### Supplementary Note D7

The image generation apparatus described in supplementary note D6, in which the medical information includes at least one selected from the group consisting of personal information regarding the patient, information on findings shown by a medical examination performed on the patient, and medical history information regarding the patient.

### Supplementary Note D8

The image generation apparatus described in any one of supplementary notes D1 to D7, in which the prediction image is used in decision making carried out by a medical service worker who provides medical examination and treatment.

### Additional Remark E

The whole or part of the example embodiments disclosed above can be described as, but not limited to, the following supplementary notes, and the present invention can be altered in various ways by a skilled person in the art within the scope of the claims

### Supplementary Note E1

A non-transitory recording medium having recorded thereon an image generation program for causing a computer to function as an image generation apparatus,
the image generation program causing the computer to carry out:
an acquiring process of acquiring a medical image of a patient;
a detecting process of detecting a lesion candidate in the medical image;
a differentiating process of generating, from information indicating the lesion candidate, a result of differentiation of the lesion candidate regarding a medical condition the patient has;
an image generating process of generating, based on the information indicating the lesion candidate and the result of differentiation of the lesion candidate, a prediction image which represents a state in which the lesion candidate will be after a lapse of time; and
an outputting process of outputting the prediction image.

### REFERENCE SIGNS LIST

1: Image generation apparatus
1A: Information processing apparatus
11, 11A: Acquiring section
12, 12A: Detecting section
13, 13A: Differentiating section
14, 14A: Image generating section
15, 50A: Outputting section
15A: Output control section

1. An image generation apparatus, comprising
at least one processor, the at least one processor carrying out:
an acquiring process of acquiring a medical image of a patient;
a detecting process of detecting a lesion candidate in the medical image;
a differentiating process of generating, from information indicating the lesion candidate, a result of differentiation of the lesion candidate regarding a medical condition the patient has;
an image generating process of generating, based on the information indicating the lesion candidate and the result of differentiation of the lesion candidate, a prediction image which represents a state in which the lesion candidate will be after a lapse of time; and
an outputting process of outputting the prediction image.

2. The image generation apparatus according to claim 1, wherein
in the image generating process, the at least one processor generates, based on the information indicating the lesion candidate and the result of differentiation of the lesion candidate, a plurality of prediction images which each represent a state in which the lesion candidate will be after a lapse of time.

3. The image generation apparatus according to claim 2, wherein
in the image generating process, the at least one processor generates respective accuracies of the plurality of prediction images; and
in the outputting process, the at least one processor outputs the plurality of prediction images and the respective accuracies of the plurality of prediction images.

4. The image generation apparatus according to claim 2, wherein
in the differentiating process, the at least one processor generates results of differentiation of a plurality of lesion candidates from the information indicating the lesion candidate; and
in the image generating process, the at least one processor generates respective prediction images for the results of differentiation of the plurality of lesion candidates.

5. The image generation apparatus according to claim 1, wherein
in the image generating process, the at least one processor generates the prediction image based on the information indicating the lesion candidate and the result of differentiation of the lesion candidate, with use of a generative model trained by machine learning in which a result of a lapse of time on a lesion is used as ground truth data, so as to generate a prediction image that represents a state in which the lesion will be after a lapse of time.

6. The image generation apparatus according to claim 5, wherein
in the acquiring process, the at least one processor acquires medical information regarding the patient in addition to the medical image; and
in the image generating process, the at least one processor generates the prediction image by inputting the information indicating the lesion candidate, the result of differentiation of the lesion candidate, and the medical information to the generative model.

7. The image generation apparatus according to claim 6, wherein
the medical information includes at least one selected from the group consisting of personal information regarding the patient, information on findings shown by a medical examination performed on the patient, and medical history information regarding the patient.

8. The image generation apparatus according to claim 1, wherein
the prediction image is used in decision making carried out by a medical service worker who provides medical examination and treatment.

9. An image generation method, comprising
at least one processor acquiring a medical image of a patient;
the at least one processor detecting a lesion candidate in the medical image;
the at least one processor generating, from information indicating the lesion candidate, a result of differentiation of the lesion candidate regarding a medical condition the patient has;
the at least one processor generating, based on the information indicating the lesion candidate and the result of differentiation of the lesion candidate, a prediction image which represents a state in which the lesion candidate will be after a lapse of time; and
the at least one processor outputting the prediction image.

10. A computer-readable non-transitory recording medium having recorded thereon a program for causing a computer to function as an image generation apparatus, the program causing the computer to carry out:
an acquiring process of acquiring a medical image of a patient;
a detecting process of detecting a lesion candidate in the medical image;
a differentiating process of generating, from information indicating the lesion candidate, a result of differentiation of the lesion candidate regarding a medical condition the patient has;
an image generating process of generating, based on the information indicating the lesion candidate and the result of differentiation of the lesion candidate, a prediction image which represents a state in which the lesion candidate will be after a lapse of time; and
an outputting process of outputting the prediction image.
